(19) United States
(12) Patent Application Publication
(10) Pub. No.: US 2021/0374873 A1
(43) Pub. Date: Dec. 2, 2021

(54) SYSTEM AND METHOD FOR CASE MANAGEMENT RISK STRATIFICATION

(71) Applicant: New Directions Behavioral Health, L.L.C., Overland Park, KS (US)

(72) Inventors: Timothy Cole, Baldwin City, KS (US); Adam Powell, Lawrence, KS (US); Elizabeth Jones, Jacksonville Beach, FL (US); Jennifer L. Baird, Chesterfield, MO (US)

(21) Appl. No.: 17/333,657

(22) Filed: May 28, 2021

Related U.S. Application Data
(60) Provisional application No. 63/037,071, filed on Jun. 10, 2020.
(60) Provisional application No. 63/032,304, filed on May 29, 2020.

(51) Int. Cl. G06Q 40/08; G16H 10/60; G16H 10/20; G06N 3/08

(52) U.S. Cl. CPC G06Q 40/08; G06N 3/08; G16H 10/20; G16H 10/60

(57) ABSTRACT

A server is programmed to predict high burden behavioral health insurance plan participants. The server receives health data from several data sources. The data is associated with the insurance plan participants. The server develops data variables and risk metrics relating to behavioral health of the participants. Sample data is collected from the health data. The sample data includes a first sample data set including a plurality of first values relating to the plurality of data variables, and a second sample data set including a plurality of second values relating to the plurality of risk metrics. The server analyzes the first and second sample data sets to determine one or more correlations between the first values and the second values. The server configures a software model to implement the correlations to predict which of the participants are at risk of becoming burden behavioral health insurance plan participants.

### CROSS-REFERENCE TO RELATED APPLICATIONS

This application claims priority from identically-titled U.S. Provisional Patent application No. 63/037,071, filed Jun. 10, 2020, and from identically-titled U.S. Provisional Patent application No. 63/032,304, filed May 29, 2020, and the entirety of each of the foregoing applications is hereby incorporated by reference herein.

### FIELD OF THE DISCLOSURE

The field of the disclosure relates generally to behavioral health case management and, more particularly, to systems and methods for predicting high burden behavioral health insurance plan participants for case management stratification.

### BACKGROUND

In the healthcare insurance industry, there are various managed care organizations that contract with insurance providers and plan sponsors to administer some part or parts of a benefits program. At least some of these managed care organizations utilize software models and software products to identify high risk members. However, these models typically focus on medical risk—for example, members with physical health conditions and risk of medical hospitalizations. While such models may factor in behavioral health claims and behavioral pharmacy claims data, they generally only identify medical risk. As such, these models may be useful in identifying total future physical health costs. These models, however, are not useful in identifying mental health and substance abuse risk due, in part, to the uniqueness and challenges of behavioral health conditions and care management.

### SUMMARY

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present disclosure will be apparent from the following detailed description of the embodiments and the accompanying drawing figures.

In one aspect, a computer-implemented method for predicting high burden behavioral health insurance plan participants is provided. The method includes receiving health data from a plurality of data sources. The health data is associated with a plurality of insurance plan participants. The method also include developing a plurality of data variables and a plurality of risk metrics relating to behavioral health based on the health data. Furthermore, the method includes collecting sample data from the health data. The sample data includes a first sample data set and a second sample data set. The first sample data set includes a plurality of first values relating to the plurality of data variables. The second sample data set includes a plurality of second values relating to the plurality of risk metrics. The method also includes analyzing the first and second sample data sets to determine one or more correlations between the first values relating to the data variables and the second values relating to the risk metrics. Moreover, the method includes configuring a software model to implement the one or more correlations to predict which of the plurality of insurance plan participants are at risk of becoming burden behavioral health insurance plan participants.

Advantages of these and other embodiments will become more apparent to those skilled in the art from the following description of the exemplary embodiments which have been shown and described by way of illustration. As will be realized, the present embodiments described herein may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of systems and methods disclosed therein. It should be understood that each figure depicts an embodiment of a particular aspect of the disclosed systems and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic of an exemplary system, in accordance with embodiments of the present invention;

FIG. 2 is an example configuration of a server for use in the system shown in FIG. 1;

FIG. 3 is an example configuration of a data source computing device for use in the system shown in FIG. 1; and

FIG. 4 is a flowchart illustrating an exemplary computer-implemented method for predicting high burden behavioral health insurance plan participants.

Unless otherwise indicated, the drawings provided herein are meant to illustrate features of embodiments of this disclosure. These features are believed to be applicable in a wide variety of systems comprising one or more embodiments of this disclosure. As such, the drawings are not meant to include all conventional features known by those of ordinary skill in the art to be required for the practice of the embodiments disclosed herein.

### DETAILED DESCRIPTION

Embodiments of the present technology relate to systems, computer-readable media, and computer-implemented methods for predicting high burden behavioral health insurance plan participants for case management stratification. Embodiments of the present technology enable case management entities an opportunity to intervene preemptively with case management services, helping save time, effort, resources, and anguish among vulnerable patient populations.

Specific embodiments of the technology will now be described in connection with the attached drawing figures. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

### Exemplary System

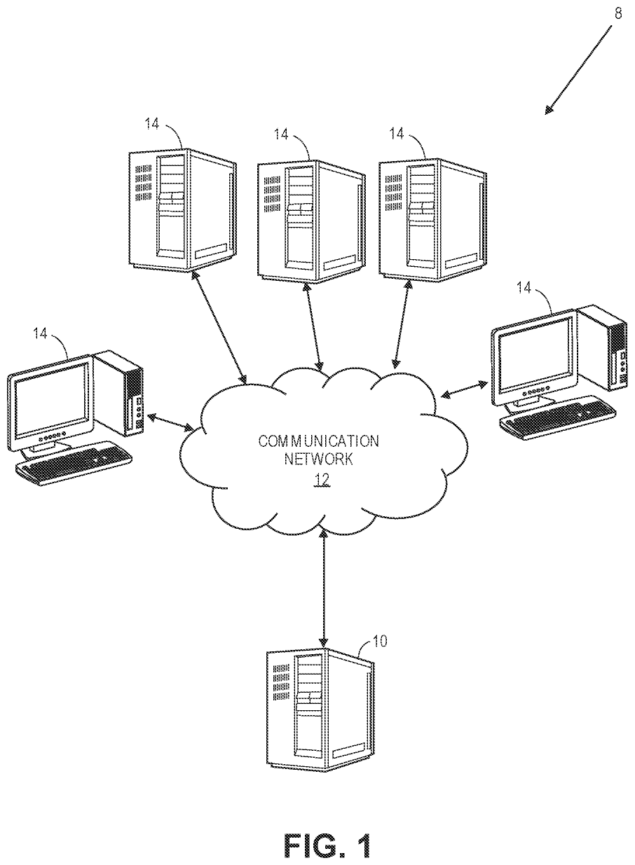

FIG. 1 depicts an exemplary system 8 in which embodiments of a server 10 may be utilized for predicting high burden behavioral health insurance plan participants. The environment may include a communication network 12 and a plurality of data source computing devices 14. Each data source computing device 14 may include a desktop computer, a laptop or tablet computer, an application server, a database server, a file server, or the like, or combinations thereof, configured to periodically or continuously provide data updates to the server 10. The server 10 may comprise and/or work in conjunction with application servers, database servers, file servers, gaming servers, mail servers, print servers, or the like, or combinations thereof. Furthermore, the server 10 may include a plurality of servers, virtual servers, or combinations thereof.

The communication network 12 may provide wired and/or wireless communication between the data source computing devices 14 and the server 10. Each of the server 10 and data source computing devices 14 may be configured to send data to and/or receive data from network 12 using one or more suitable communication protocols, which may be the same communication protocols or different communication protocols as one another.

The communication network 12 generally allows communication between the data source computing devices 14 and the server 10. For example, the data source computing devices 14 may, upon request, periodically and/or continuously push or otherwise provide new or updated data regarding plan participants to the server 10 over the communication network 12.

Network 12 may include one or more telecommunication networks, nodes, and/or links used to facilitate data exchanges between one or more devices and may facilitate a connection to the Internet for devices configured to communicate with network 12. The communication network 12 may include local area networks, metro area networks, wide area networks, cloud networks, the Internet, cellular networks, plain old telephone service (POTS) networks, and the like, or combinations thereof.

The communication network 12 may be wired, wireless, or combinations thereof and may include components such as modems, gateways, switches, routers, hubs, access points, repeaters, towers, and the like. The data source computing devices 14 and server 10 may connect to the communication network 12 either through wires, such as electrical cables or fiber optic cables, or wirelessly, such as radio frequency (RF) communication using wireless standards such as cellular 2G, 3G, 4G or 5G, Institute of Electrical and Electronics Engineers (IEEE) 802.11 standards such as WiFi, IEEE 802.16 standards such as WiMAX, Bluetooth™, or combinations thereof. In aspects in which network 12 facilitates a connection to the Internet, data communications may take place over the network 12 via one or more suitable Internet communication protocols. For example, network 12 may be implemented as a wireless telephony network (e.g., GSM, CDMA, LTE, etc.), a Wi-Fi network (e.g., via one or more IEEE 802.11 Standards), a WiMAX network, a Bluetooth network, etc.

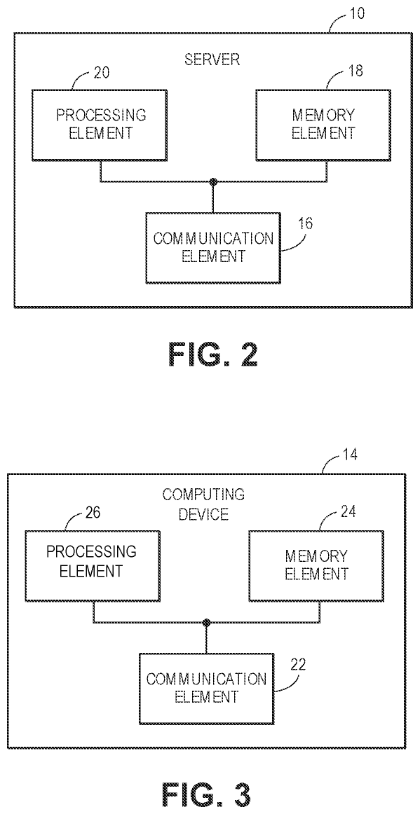

The server 10 generally retains electronic data and may respond to requests to retrieve data, as well as to store data. The server 10 may be configured to include or execute software, such as file storage applications, database applications, email or messaging applications, web server applications, and/or high burden participant prediction software or the like. As indicated in FIG. 2, the server 10 may broadly comprise a communication element 16, a memory element 18, and a processing element 20. Likewise, as indicated in FIG. 3, each of the data source computing devices 14 may broadly comprise a communication element 22, a memory element 24, and a processing element 26.

The communication elements 16, 22 each generally allows communication with external systems or devices, including network 12, such as via wireless communication and/or data transmission over one or more direct or indirect radio links between devices. The communication elements 16, 22 each may include signal or data transmitting and receiving circuits, such as antennas, amplifiers, filters, mixers, oscillators, digital signal processors (DSPs), and the like. The communication elements 16, 22 each may establish communication wirelessly by utilizing RF signals and/or data that comply with communication standards such as cellular 2G, 3G, or 4G, WiFi, WiMAX, Bluetooth™, and the like, or combinations thereof. In addition, the communication elements 16, 22 each may utilize communication standards such as ANT, ANT+, Bluetooth™ low energy (BLE), the industrial, scientific, and medical (ISM) band at 2.4 gigahertz (GHz), or the like.

Alternatively, or in addition, the communication elements 16, 22 each may establish communication through connectors or couplers that receive metal conductor wires or cables which are compatible with networking technologies, such as ethernet. In certain embodiments, the communication elements 16, 22 each may also couple with optical fiber cables. The communication elements 16, 22 each may be in communication with corresponding ones of the processing elements 20, 26 and the memory elements 18, 24, via, e.g., wired or wireless communication.

The memory elements 18, 24 each may include electronic hardware data storage components such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the memory elements 18, 24 each may be embedded in, or packaged in the same package as, the corresponding one of the processing elements 20, 26. The memory elements 18, 24 each may include, or may constitute, a “computer-readable medium.” The memory elements 18, 24 each may store the instructions, code, code segments, software, firmware, programs, applications, apps, modules, agents, services, daemons, or the like that are executed by the processing elements 20, 26, including—in the case of processing element 20 and the memory element 18—the high burden participant prediction software or the like. The memory elements 18, 24 each may also store settings, data, documents, sound files, photographs, movies, images, databases, and the like, including the items described throughout this disclosure.

The processing elements 20, 26 each may include electronic hardware components such as processors. The processing elements 20, 26 each may include digital processing unit(s). The processing elements 20, 26 each may include microprocessors (single-core and multi-core), microcontrollers, digital signal processors (DSPs), field-programmable gate arrays (FPGAs), analog and/or digital application-specific integrated circuits (ASICs), or the like, or combinations thereof. The processing elements 20, 26 each may generally execute, process, or run instructions, code, code segments, software, firmware, programs, applications, apps, modules, agents, processes, services, daemons, or the like, including—in the case of processing element 20—the high burden participant prediction software described throughout this disclosure. The processing elements 20, 26 each may also include hardware components such as finite-state machines, sequential and combinational logic, and other electronic circuits that can perform the functions necessary for the operation of the current invention. The processing elements 20, 26 each may be in communication with the other electronic components through serial or parallel links that include address busses, data busses, control lines, and the like.

Through hardware, software, firmware, or combinations thereof, the processing elements 20, 26 each may be configured or programmed to perform the functions described hereinbelow.

### Exemplary Computer-Implemented Method

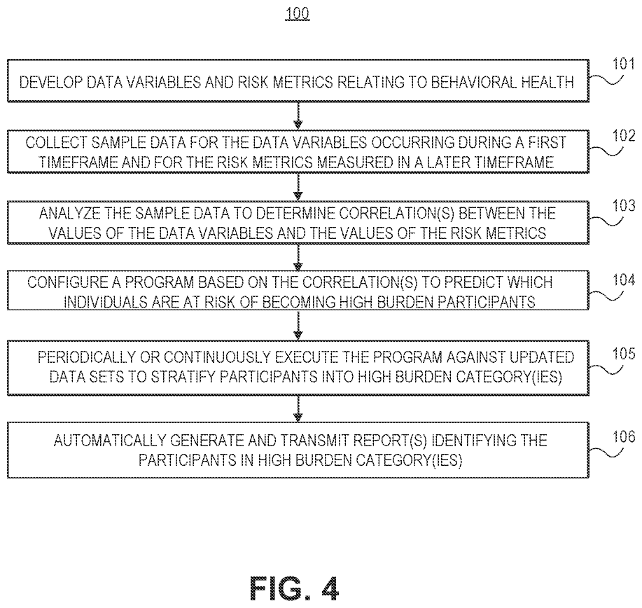

FIG. 4 depicts a listing of steps of an exemplary computer-implemented method 100 for predicting high burden behavioral health insurance plan participants. The steps may be performed in the order shown in FIG. 4, or they may be performed in a different order. Furthermore, some steps may be performed concurrently as opposed to sequentially. In addition, some steps may be optional.

The computer-implemented method 100 is described below, for ease of reference, as being executed by exemplary devices and components introduced with the embodiments illustrated in FIGS. 1-3. For example, the steps of the computer-implemented method 100 may be performed by the data source computing devices 14, the server 10 and the network 12 through the utilization of processors, transceivers, hardware, software, firmware, or combinations thereof. However, a person having ordinary skill will appreciate that responsibility for all or some of such actions may be distributed differently among such devices or other computing devices without departing from the spirit of the present invention. For example, the steps performed by the server(s) 10 may be performed in whole or in part by one or more other computing devices—such as laptop or desktop computers or other personal computing devices—without departing from the spirit of the present invention.

One or more computer-readable medium(s) may also be provided. The computer-readable medium(s) may include one or more executable programs stored thereon, such as the high burden participant prediction software, wherein the program(s) instruct one or more processing elements to perform all or certain of the steps outlined herein. The program(s) stored on the computer-readable medium(s) may instruct the processing element(s) to perform additional, fewer, or alternative actions, including those discussed elsewhere herein.

Referring to step 101, data variables and risk metrics relating to behavioral health may be developed. In an embodiment, the data variables may comprise a series of structured data types that plausibly correlate, positively or negatively, with increased risk metrics. In an embodiment, the risk metrics may comprise one or more data metrics or like indicators that an individual plan participant is a high burden on the plan.

For example, a risk metric may comprise high total behavioral healthcare costs (e.g., costs assessed to a corresponding health insurance plan) paid out in a subsequent timeframe on behalf of a plan participant, and/or a threshold such as admission of the plan participant to an inpatient residential treatment center during the subsequent timeframe. Predictive data variables may relate to types of events occurring in a preceding timeframe (i.e., before the subsequent timeframe), and may comprise: allowed behavioral health costs for the plan participant; inpatient admissions of the plan participant; residential treatment center admissions of the plan participant; and/or number of diagnosed behavioral health diagnosis groups for the plan participant. Moreover, one or more of the predictive data variables may also (more broadly) relate to events outside of the preceding timeframe, such as where lifetime inpatient residential trademark center admissions of the plan participant are considered.

The data for the variables and the risk metrics may originate with a plurality of data sources dispersed across a plurality of systems. For example, the data sources may comprise proprietary or public databases, and may be derived from behavioral health data and/or medical data comprising historical claims data, authorizations data, eligibility data, pharmacy data, clinical data, case management data, facility-provided data and/or other types of behavioral health data. In an embodiment, the data sources may comprise a pharmacy server, a hospital facility server, a proprietary database server, a case management server and/or other data sources controlled by multiple entities.

In a preferred embodiment, another source of data may comprise questionnaires that are automatically generated with structured data fields in which healthcare providers and/or plan participants may enter additional data. For example, a case management server of embodiments of the present invention may automatically identify—e.g., from claims data, authorizations data, hospital data or the like—that a plan participant has interacted with a behavioral health professional (i.e., at a facility or remotely) within a given timeframe. The server may automatically populate and transmit a questionnaire comprising pre-determined structured data inquiries seeking input from the professional and/or the participant about the interaction. The professional and/or participant may receive respective questionnaires (e.g., at a computing device via e-mail or internet-accessible portal), respond to the inquiries with input, and provide the input to the server. The server may thereafter incorporate the input into a database for storing participant data for analysis according to the description below.

It should also be noted that the data variables selected for analysis may require enrichment relative to data made available from the data sources and outlined above. For example, in an embodiment, the data provided to the case management server (or other computing device performing analysis outlined below) may be relatively raw and may be enriched to more meaningful forms via consolidation, manipulation, combination, or the like. In an embodiment, the data sources may provide raw timestamped data for a plurality of plan participants regarding, for instance, each participant's historical behavioral healthcare costs, inpatient admissions, diagnoses, census scores, and the like. Enrichment of the raw data may include culling data relating to events not occurring during a pre-determined preceding timeframe, summing events or costs occurring during the preceding timeframe across one or more classes (e.g., summing all events of the same type or of multiple types within a category), or the like.

Types of raw data that may be made available by the data sources may include timestamped data comprising: diagnosis, procedure, and National Drug Codes; information on service provider; prescribing physician; health plan payments; participant payment responsibility; type and date of bill paid; facility type; revenue codes; service dates; encrypted SSN or participant identification number (or other personal identifier for plan participants); type of product (HMO, POS, indemnity, etc.); type of contract (single person, family, etc.); patient demographics (date of birth, gender, ZIP code); administrative fees; back end settlement amounts; referrals; test results from lab work, imaging, etc.; provider affiliation with group practice; provider networks; denied claims; workers' compensation claims; premium information; and/or capitation fees.

Further, as noted above, exemplary data variables incorporated or based on raw data types may include: lifetime inpatient and/or residential treatment center admissions; gaps in behavioral health care; barriers in social determinants of health; number of behavioral health diagnoses in the preceding timeframe; inpatient admissions during the preceding timeframe; census score during the preceding timeframe; number of substance use diagnoses during the preceding timeframe; total behavioral health costs during the preceding timeframe (other than for applied behavioral analysis); total number of behavioral health-related emergency room visits during the preceding timeframe; number of rapid readmissions during the preceding timeframe; number of residential treatment center admissions during the preceding timeframe; number of behavioral health providers during the preceding timeframe; number of behavioral health visits during the preceding timeframe; number of bipolar disorder diagnoses during the preceding timeframe, and/or other data variables that may be correlated with risk metrics.

Other enriched data variables are outlined in the following Table 1:

TABLE 1

| | Variable | | |
|---|---|---|---|
| Variable | Type | Definition | Data Source |
| Total behavioral | Claims Costs | Preceding timeframe | Claims |
| health Costs | | behavioral health Costs | |
| (Unrelated to | | (allowed)-total costs for | |
| applied | | behavioral health primary | |
| behavioral | | Diagnosis-Excludes | |
| analysis) | | Prescriptions; Preceding | |
| | | timeframe with 3 month | |
| | | lag; excludes Autism | |
| | | applied behavioral analysis | |
| | | costs | |
| Recent | Claims Costs | behavioral health costs | Claims |
| behavioral | | (allowed) for primary | |
| health Costs | | behavioral health | |
| | | Diagnosis in past 3 | |
| | | months, no lag | |
| Behavioral | Claims | Count of primary | Claims |
| health | Diagnoses; if | behavioral health diagnosis | |
| Diagnosis | no claims, | groups in preceding | |
| Groups | used | timeframe | |
| | authorizations | | |
| Behavioral | Claims | Flags for presence/absence | Claims |
| health | Diagnoses; if | of primary or secondary | |
| Diagnosis | no claims, | Diagnosis for major | |
| Flags | used | depression, bipolar, | |
| | authorizations | schizophrenia, autism, | |
| | | eating disorder, anxiety | |
| | | disorder, opioid use, | |
| | | alcohol use, other | |
| | | substance use in Preceding | |
| | | timeframe | |
| Substance Use | Claims | Count of distinct Substance | Claims |
| Disorder | Diagnoses; if | Use Disorder diagnosis | |
| Diagnosis | no claims, | groups in preceding | |
| Groups | used | timeframe (e.g., Opioids, | |
| | authorizations | Inhalants, Alcohol, | |
| | | Cocaine, etc.) | |
| Suicide | Claims | Primary or secondary | Claims |
| Diagnosis | Diagnoses; if | suicide attempt diagnosis | |
| Indicator | no claims, | in preceding timeframe | |
| | used | | |
| | authorizations | | |
| behavioral | Claims | Preceding timeframe | Claims |
| health | Utilization | Emergency Room visits | |
| Emergency | | with a primary behavioral | |
| Room Visits | | health Diagnosis | |
| behavioral | Claims | Preceding timeframe | Claims |
| health | Utilization | distinct provider count for | |
| Professional | | professional behavioral | |
| Provider Count | | health providers | |
| behavioral | Claims | Count of behavioral health | Claims |
| health | Utilization | professional visits in | |
| Professional | | preceding timeframe | |
| Visit Count | | | |
| Auth Higher | Auth | Auth based weighted # of | Auths |
| Level of Care | Utilization | admits for higher levels of | |
| Admits | | care (1*Acute inpatient | |
| | | Admits + .75*Residential | |
| | | Admits + .5*Partial | |
| | | Hospitalization | |
| | | Admits + .25*Intensive | |
| | | Outpatient Admits − all | |
| | | preceding timeframe) | |
| Child | Auth | Participant who is admitted | Auths |
| Admission | Utilization | to inpatient or residential | |
| Indicator | | treatment and is under 18 | |
| | | years old on the date of | |
| | | admission | |
| Eating Disorder | Auth | Participant who is admitted | Auths |
| Admission | Utilization | to inpatient or residential | |
| Indicator | | treatment and has an active | |
| | | eating disorder diagnosis | |
| Frequent | Auth | Participant has at least 2 | Auths |
| Readmission | Utilization | behavioral health acute | |
| Indicator | | inpatient or residential | |
| | | admissions in a 60 day | |
| | | period in the preceding | |
| | | timeframe | |
| Lifetime | Auth | Lifetime count of | |
| Inpatient and | Utilization | behavioral health inpatient | Auths |
| Residential | | and residential admissions | |
| Treatment | | | |
| Admissions | | | |
| Acute Inpatient | Claims | Count of Acute behavioral | Claims |
| Admits Count | Utilization | health inpatient Admits in | |
| | | Preceding timeframe | |
| Residential | Claims | Count of residential | Claims |
| Admit Count | Utilization | treatment center Admits in | |
| | | Preceding timeframe | |
| Partial Hospital | Claims | Count of partial | Claims |
| Admit Count | Utilization | hospitalization program | |
| | | Admits in Preceding | |
| | | timeframe | |
| Intensive | Claims | Count of intensive | Claims |
| Outpatient | Utilization | outpatient program Admits | |
| Admit Count | | in Preceding timeframe | |
| Socioeconomic | Clinical | 1/0 flag if participant has a | Clinical |
| Diagnosis | | primary or secondary | Assessment |
| Indicator | | Diagnosis code considered | (case |
| | | a socioeconomic risk factor | manage- |
| | | OR of the participants with | ment |
| | | an substance abuse acute | clinical |
| | | inpatient or residential | system) |
| | | admit in past 30 days, were | |
| | | there barriers noted in one | |
| | | of the clinical assessments | |
| | | (includes the following | |
| | | barriers: No support | |
| | | system, Transportation, | |
| | | Cultural, Other barriers, | |
| | | Financial, Housing, | |
| | | Occupational, Access to | |
| | | providers, Participant is | |
| | | not adherent with | |
| | | medication/treatment, | |
| | | Cultural/Linguistic, | |
| | | Language) | |
| Medication | Participant | 1/0 flag educated on MAT | Clinical |
| Assisted | risk data | but not discharged on | Assessment |
| Treatment | | MAT med | (case |
| (MAT) | | | manage- |
| Discharge Risk | | | ment |
| | | | clinical |
| | | | system) |
| MAT | Participant | Based on discharge review, | Clinical |
| Education | risk data | 1/0 flag if they were | Assessment |
| Risk | | supposed to be educated | (case |
| Diagnostic | | on MAT but were not | manage- |
| | | | ment |
| | | | clinical |
| | | | system) |
| Trigger | | The participant had a | |
| Precondition | | behavioral health (BH) | |
| | | service within the past 90 | |
| | | days (of any type) and | |
| | | satisfies one of the | |
| | | following Diagnostic | |
| | | Triggers A-E. | |
| Diagnostic | | The participant had an | |
| Trigger A | | eating disorder admission | |
| | | during the preceding 12 | |
| | | months | |
| Diagnostic | | The participant had a | |
| Trigger B | | suicide or self-harm | |
| | | primary or secondary | |
| | | diagnosis during the | |
| | | preceding 12 months | |
| Diagnostic | | The participant had a | |
| Trigger C | | postpartum depression | |
| | | primary or secondary | |
| | | diagnosis during the | |
| | | preceding 12 months | |
| Diagnostic | | The participant had a | |
| Trigger D | | psychotic disorder primary | |
| | | or secondary diagnosis | |
| | | between the ages of 14 and | |
| | | 26 and has never had a | |
| | | higher level of care | |
| | | admission | |
| Diagnostic | | The participant had an | |
| Trigger E | | opioid use disorder | |
| | | primary diagnoses and at | |
| | | least one inpatient and/or | |
| | | residential treatment center | |
| | | admission during the | |
| | | preceding 90 days or one | |
| | | emergency room visit in | |
| | | the preceding timeframe | |
| Discharge | | Acute psychiatric inpatient | |
| Issues | | discharge in past 30 days | |
| | | with no scheduled follow- | |
| | | up appointment, or Acute | |
| | | inpatient discharge in past | |
| | | 30 days with barriers noted | |
| | | in clinical assessment, or a | |
| | | substance use disorder | |
| | | discharge in past 30 days | |
| | | with no medication | |
| | | assisted treatment | |
| | | education, or with no | |
| | | medication assisted | |
| | | treatment meds if educated | |
| Admit Recency | | Psychiatric Inpatient or | |
| | | Residential admission | |
| | | within the last 30 days and | |
| | | not currently enrolled in | |
| | | case management | |
| Case | | Whether or not the | |
| management | | participant is currently | |
| status | | assigned a case manager | |
| Rising Risk | | Regardless of claims lag, if | |
| | | a participant has incurred | |
| | | >=$4000 in claims costs in | |
| | | past 4 months and has had | |
| | | no contact in the past 90 | |
| | | days | |
| Acute Inpatient | Auth | Participant has been | Auths |
| or Residential | Utilization | admitted to Acute inpatient | |
| in Past | | or residential treatment | |
| 30 Days Flag | | center in Past 30 days | |
| | | (approved or denied) | |
| Last Case | CM Data | The last CM referral and | CM |
| Management | | enrollment dates for | Program |
| (CM) Referral | | participant | Data (case |
| or Enrollment | | | manage- |
| | | | ment |
| | | | clinical |
| | | | system) |
| Discharged with | Auth | Participant has been | Auths |
| no scheduled | Utilization | discharged to Acute | |
| appointment | | Inpatient Past 30 days | |
| date | | (approved or denied) and | |
| | | has no scheduled | |
| | | appointment date entered | |

It should be noted that, in one or more embodiments, only data that is unrelated to applied behavioral analysis is considered in connection with the variables listed in Table 1.

Referring to step 102, sample data may be collected for the data variables and the risk metrics. The sample data may be collected from the data sources in raw and/or finished form with respect to the correlation analysis outlined below. Raw data may be further processed to place it in finished form for such analysis, as outlined above, such as by culling sample data not occurring in the corresponding timeframe. In an embodiment, sample data corresponding to the data variables is culled to include only data relating to events occurring within a predefined preceding timeframe (e.g., a first year), and the sample data corresponding to the risk metrics is culled to include only data relating to events occurring within a predefined subsequent timeframe (e.g., a second year immediately following the first year).

Moreover, it should be noted that sample data is preferably keyed to individual participants—whether in anonymized or personally identifiable records. That is, the sample data for data variables and risk metrics is preferably identified with unique identifiers (again, whether anonymized, pseudo-anonymized, or personally identifiable) that allow the sample data for respective plan participants to be analyzed to determine correlations between data variables and risk metrics.

Referring to step 103, the sample data may be analyzed to determine one or more correlation(s) between the values of the data variables and the risk metrics. In an embodiment, a high burden participant prediction software program performs such an analysis. For example, the program may comprise a machine learning program or technique. The program may scrutinize the sample data using one or more machine learning techniques to generate one or more correlations or other relational observations. The program and/or machine learning program(s) may therefore recognize or determine patterns relating observed participant behavior in a first time period (i.e., the preceding timeframe) to behavior or events in a second time period (i.e., the subsequent timeframe). The machine learning techniques or programs may include curve fitting, regression model builders, convolutional or deep learning neural networks, combined deep learning, pattern recognition, or the like. Based upon this data analysis, the program and/or machine learning program(s) may be configured to predict high burden participants, as discussed in more detail below.

In supervised machine learning, the program may be provided with example inputs (i.e., sample data relating to the data variables) and their associated outputs (i.e., sample data for the risk metrics), and may seek to discover a general rule that maps inputs to outputs, so that when subsequent novel inputs are provided the processing element may, based upon the discovered rule, accurately predict the correct output. In unsupervised machine learning, the program may be required to find its own structure in unlabeled example inputs.

The program may utilize classification algorithms such as Bayesian classifiers and decision trees, sets of pre-determined rules, and/or other algorithms to predict high burden participants based on behavior in a given timeframe. Moreover, in a preferred embodiment, different and/or separately-trained algorithms and models are optimized for use in detecting respective types of anomalous behaviors.

In an example, data regression may be used to correlate data variable values (see examples above) with risk metric values (see examples above) across a participant population. The output may comprise one or more tables listing the data variables in order of descending correlation coefficient values with respect to respective risk metrics.

Referring to step 104, a program may be configured based on the correlation(s) to predict which individuals are at risk of becoming high burden participants. In an embodiment, the program is primarily configured in connection with the analysis step of 103, such as where a deep learning neural network is trained on the sample data in the analysis step, and thereby configured to receive novel input and provide such a prediction as output. In another embodiment, the correlation(s) are rawer and must be built into a super structure of the program to configure it for such predictions. For example, the exemplary tables of correlation coefficients outlined above may be combined into one or more weighted summations for use alone or in combination to generate output scores for each participant.

In an embodiment, correlation coefficient tables may be combined into a weighted summation, the weightings being based on the correlation coefficients. Depending on the variables used, the program may be further configured to normalize or otherwise manipulate outputs from the weighted summation and convert to a more useable scale or format. For example, a weighted summation may have a maximum output of eighty-eight (88), and the program may be configured to convert the raw output to a new scale of zero to ten (0-10). Moreover, the program may be configured to map values along the finished scale to automated actions (such as populating a report including or prioritizing participants with certain values along the finished scale).

The program may additionally be configured with rules and/or a decision tree directed toward eliminating participants meeting pre-defined criteria from consideration. For example, the program may be configured only to generate output scores for participants that had a primary behavioral health-related claim, authorization, or diagnosis within a pre-defined lookback window (e.g., the preceding twelve (12) months). For another example, only participants that were eligible under the health plan in question for a minimum threshold proportion of the pre-defined lookback window may be considered.

It should also be noted that one or more data variables may be excluded from consideration in the program. For example, the one or more data variables may be inconsistently correlated with corresponding risk metrics and/or may be loosely correlated therewith, and therefore may be excluded from consideration in the program.

Referring to step 105, the program may be executed against updated or new data sets to stratify participants into high burden category(ies). In an embodiment, the program is executed periodically and/or continuously (e.g., based on triggers, pre-defined passage of time, or processor availability) against updated data sets for participants meeting threshold consideration requirements such as those outlined above. The updated data sets may be partially or entirely provided by external data sources, and such data may be updated at varying intervals. Moreover, the data sets may be considered by the program on a rolling basis, i.e., during a trailing time period measured from the time of calculation (i.e., only data relating to events occurring within the preceding twelve (12) months are considered).

Stratification of participants by the program may include translating raw outputs to a finished output scale (as noted above) and/or combining the numerical outputs with business rules or decision trees. For example, a numerical output (e.g., “points levels” based on the weighted summation(s) discussed above) may be combined with one or more business rules (also based on data variables) to generate the stratification schema outlined below in Table 2:

TABLE 2

| | | PRIORITY |
|---|---|---|
| RISK TYPE | RISK TYPE DEFINITION | CATEGORY |
| CURRENT | Participant admitted to inpatient and/or | URGENT |
| RISK | residential treatment center in past 30 | |
| | days AND Prior Year Spend >= $30K | |
| | or Points Level >= 6 | |
| CONTIN- | Prior year and current year allowed | ROUTINE |
| UOUSLY | costs both $30k+ | |
| HIGH RISK | | |
| HIGH RISK | Points level >= 9 | ROUTINE |
| CURRENT | Participant admitted to inpatient and/or | URGENT |
| RISK | residential treatment center in past 30 | |
| CURRENTLY | days AND currently enrolled in either | |
| ENROLLED | care solutions, care transitions or | |
| CM | participant care link | |
| MEDIUM | Participant admitted to inpatient and/or | URGENT |
| RISK NO | residential treatment center in past 30 | |
| FOLLOW | days AND there is no scheduled | |
| UP APPT | appointment date entered. | |
| RISING | Participant has spent >=6000 in past | ROUTINE |
| RISK | 90 days (no lag) | |
| MEDIUM | Participant spent >=$100k in current | ROUTINE |
| RISK HIGH | year | |
| COSTS | | |
| MEDIUM | Participant has Points Level of 7 or 8 | ROUTINE |
| RISK | and a socioeconomic/other barrier | |
| BARRIERS + | entered in a survey or the like | |
| POINTS | | |
| MEDIUM | Participant admitted for SUBSTANCE | ROUTINE |
| RISK MAT | USE DISORDER to inpatient and/or | |
| | residential treatment center in past 30 | |
| | days AND had neither been MAT | |
| | educated nor discharged on MAT | |
| MEDIUM | Participant admitted to inpatient and/or | ROUTINE |
| RISK | residential treatment center in past 30 | |
| BARRIERS + | days AND had a socioeconomic/other | |
| RECENT | barrier entered in a survey or the like | |
| ADMIT | | |
| LOW RISK | All other participants in selected | ROUTINE |
| | population | |
| TOTAL | ALL | ALL |

Another example of a combined schema (again, incorporating data variables outlined above) is set out below in Table 3:

TABLE 3

| | Detailed | | | | |
|---|---|---|---|---|---|
| Broad Risk | Risk | Risk | Discharge | Admit | CM |
| Category | Category | Points | Issues | Recency | Status |
| Priority | Recent & | High | No | Recent | Not |
| Members | High | | Discharge | | Assigned |
| | | | Issues | | |
| Priority | High | High | No | Not Recent | Not |
| Members | | | Discharge | | Assigned |
| | | | Issues | | |
| Priority | Recent & | High | No | Recent | Assigned |
| Members | High- | | Discharge | | |
| | Assigned | | Issues | | |
| Priority | Recent & | Moderate | No | Recent | Assigned |
| Members | Moderate + | | Discharge | | |
| | Diagnostic | | Issues | | |
| | Trigger- | | | | |
| | Assigned | | | | |
| Priority | Recent & | Any | Discharge | Recent | Assigned |
| Members | Discharge | | Issues | | |
| | Issues- | | | | |
| | Assigned | | | | |
| Priority | Recent & | Low | No | Recent | Assigned |
| Members | Low + | | Discharge | | |
| | Diagnostic | | Issues | | |
| | Trigger- | | | | |
| | Assigned | | | | |
| Priority | Recent | Low To | No | Recent | Assigned |
| Members | Low & | Moderate | Discharge | | |
| | Moderate- | | Issues | | |
| | Assigned | | | | |
| Priority | Recent & | Moderate | No | Recent | Not |
| Members | Moderate + | | Discharge | | Assigned |
| | Diagnostic | | Issues | | |
| | Trigger | | | | |
| Priority | Recent & | Any | Discharge | Recent | Not |
| Members | Discharge | | Issues | | Assigned |
| | Issues | | | | |
| Priority | Recent & | Low | No | Recent | Not |
| Members | Low + | | Discharge | | Assigned |
| | Diagnostic | | Issues | | |
| | Trigger | | | | |
| Rising Risk | Moderate | Moderate | No | Not Recent | Not |
| Members | & Rising + | | Discharge | | Assigned |
| | Diagnostic | | Issues | | |
| | Trigger | | | | |
| Rising Risk | Moderate | Moderate | No | Not Recent | Not |
| Members | & Rising | | Discharge | | Assigned |
| | | | Issues | | |
| Rising Risk | Low & | Low | No | Not Recent | Not |
| Members | Rising + | | Discharge | | Assigned |
| | Diagnostic | | Issues | | |
| | Trigger | | | | |
| At-Risk | Moderate | Moderate | No | Not Recent | Not |
| Members | | | Discharge | | Assigned |
| | | | Issues | | |
| At-Risk | Recent | Low to | No | Recent | Not |
| Members | Low & | Moderate | Discharge | | Assigned |
| | Moderate | | Issues | | |
| Low Risk | Minimal | Minimal | No | Not Recent | Not |
| Members | | | Discharge | | Assigned |
| | | | Issues | | |
| Healthy | Negligible | Negligible | No | Not Recent | Not |
| Members | | | Discharge | | Assigned |
| | | | Issues | | |

One of ordinary skill will appreciate that the examples set out above are merely illustrative, and that a variety of data variables and schema may be adopted without departing from the spirit of the present invention.

Referring to step 106, a report may be generated to identify those participants which fall within high burden categories. In an embodiment, each time participants are ranked or stratified within a schema outlined above, a report may be generated to identify those predicted to be high burden in the future. “High,” in this context, may mean a level of burden a case management service or health plan considers to be sufficient to justify or compel inclusion in a case management plan or service. For example, each participant whose data places him or her into a “Priority,” “High Risk,” or “Current Risk” category may be included and/or emphasized in the report. The report may be automatically e-mailed or otherwise transmitted to a pre-determined list of individuals employed or contracted by the case management service or health plan.

A case management service or health plan may utilize the report(s) to identify participants that should be contacted to determine willingness to receive case management services.

The method 100 may include additional, less, or alternate actions, including those discussed elsewhere herein such as in the section entitled “Exemplary System,” and/or may be implemented via a computer system, communication network, one or more processors or servers, and/or computer-executable instructions stored on non-transitory storage media or computer readable medium.

### ADDITIONAL CONSIDERATIONS

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current technology can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order recited or illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises a general-purpose processor configured using software, the general-purpose processor may be configured as respective different processing elements at different times. Software may accordingly configure the processing element to constitute a particular hardware configuration at one instance of time and to constitute a different hardware configuration at a different instance of time.

Computer hardware components, such as communication elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element-implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

We claim:

1. A computer-implemented method for predicting high burden behavioral health insurance plan participants comprising, via one or more processors:
receiving health data from a plurality of data sources, the health data being associated with a plurality of insurance plan participants;
developing a plurality of data variables and a plurality of risk metrics relating to behavioral health based on the health data;
collecting sample data from the health data, the sample data including a first sample data set and a second sample data set, the first sample data set including a plurality of first values relating to the plurality of data variables, the second sample data set including a plurality of second values relating to the plurality of risk metrics;
analyzing the first and second sample data sets to determine one or more correlations between the first values relating to the data variables and the second values relating to the risk metrics; and
configuring a software model to implement the one or more correlations to predict which of the plurality of insurance plan participants are at risk of becoming burden behavioral health insurance plan participants.

2. The computer-implemented method in accordance with claim 1,
the analyzing operation comprising supervised training of a machine learning program stored in a memory element of a server using the first sample data set as example input data and the second sample data set as example output data.

3. The computer-implemented method in accordance with claim 2, wherein
the machine learning program determines, based on the supervised machine learning training, one or more general rules that maps input data to output data.

4. The computer-implemented method in accordance with claim 2,
the machine learning program comprising one or more of the following: curve fitting, regression model builders, convolutional neural networks, deep learning neural networks, combined deep learning, and pattern recognition techniques.

5. The computer-implemented method in accordance with claim 1,
the analyzing operation comprising analyzing, using data regression, to determine the one or more correlations between the first values and the second values.

6. The computer-implemented method in accordance with claim 5,
the analyzing operation further comprising providing an output including one or more tables listing the plurality of data variables in an order of descending correlation coefficient values with respect to the respective plurality of risk metrics.

7. The computer-implemented method in accordance with claim 6,
the configuring operation comprising combining the one or more tables into one or more weighted summations for use in generating one or more output scores for one or more of the insurance plan participants.

8. The computer-implemented method in accordance with claim 7,
the one or more weighted summations including a sum of weighting values, the weighting values being based on the correlation coefficient values.

9. The computer-implemented method in accordance with claim 7, wherein
the output scores are transformed using one of a normalization operation or a scaling operation.

10. The computer-implemented method in accordance with claim 7,
the configuring operation further comprising configuring the software model to implement one or more of the following: one or more rules and a decision tree.

11. The computer-implemented method in accordance with claim 10, wherein
each of the implemented one or more rules and decision tree is configured to eliminate one or more of the insurance plan participants meeting a pre-defined criteria from consideration as being at risk of becoming burden behavioral health insurance plan participants.

12. The computer-implemented method in accordance with claim 11, wherein
the software model is configured to implement the one or more rules or decision tree to generate one or more output scores only for the insurance plan participants associated with health data meeting a first criterion relating to a selected data variable of the plurality of data variables.

13. The computer-implemented method in accordance with claim 1,
the collecting operation comprising culling the first sample data set to include only portions of the health data relating to events occurring within a predefined preceding timeframe.

14. The computer-implemented method in accordance with claim 13,
the collecting operation comprising culling the second sample data set to include only portions of the health data relating to events occurring within a predefined subsequent timeframe, the subsequent timeframe being a period following at least a portion of the predefined preceding timeframe.

15. The computer-implemented method in accordance with claim 1,
the operation of developing comprising developing a plurality of predictive data variables that positively or negatively correlate to increased risk metrics.

16. The computer-implemented method in accordance with claim 1, wherein
the plurality of data sources are dispersed across a plurality of systems.

17. The computer-implemented method in accordance with claim 16,
the plurality of data sources including one or more of proprietary databases and public databases.

18. The computer-implemented method in accordance with claim 16,
the plurality of data sources including one or more of the following: a pharmacy server, a hospital facility server, a proprietary database server, and a case management server.

19. The computer-implemented method in accordance with claim 1,
the plurality of data sources including one or more questionnaires automatically generated with structured data fields for receiving additional data, each of the one or more questionnaires being generated by a case management server that automatically identifies that a respective one of the plurality of insurance plan participants has interacted with a behavioral health professional within a predetermined timeframe, the case management server transmitting the respective questionnaire to a computing device associated with the behavioral health professional.

20. The computer-implemented method in accordance with claim 1,
the health data including one or more of the following: historical claims data, authorizations data, eligibility data, pharmacy data, clinical data, case management data, and facility-provided data.
